(19) United States
(12) Patent Application Publication
(10) Pub. No.: US 2012/0220798 A1
(43) Pub. Date: Aug. 30, 2012

(54) METHOD FOR CARBOXYLIC ACID ESTERIFICATION

(75) Inventors: Daniel Amoros, Venissieux (FR); Pascal Pitiot, Lyon (FR); Mathias Brehelin, Lyon (FR); Alexandre Tresmondi, Campinas (BR)

(73) Assignee: RHODIA OPERATIONS, Aubervilliers (FR)

(21) Appl. No.: 13/392,218

(22) PCT Filed: Aug. 23, 2010
(86) PCT No.: PCT/EP2010/062265

§ 371 Date: May 11, 2012

(30) Foreign Application Priority Data
Aug. 24, 2009 (FR) 09 55762

(51) Int. Cl. C07C 67/08

(52) U.S. Cl. 560/265

(57) ABSTRACT

The present invention relates to a method for esterification from alcohols and carboxylic acids using a heterogeneous catalyst, wherein the esterification reaction is performed at high temperatures enabling an improvement in the compromise between selectivity, conversion, and kinetics in various types of devices for implementing the method.

The present invention relates to an esterification process starting from alcohols and carboxylic acids in heterogeneous catalysis in which the esterification reaction is carried out at high temperatures, making it possible to increase the compromise in selectivity, in conversion and in kinetics in various types of devices for the implementation of the process.

### PRIOR ART

It is known to manufacture carboxylic esters by the esterification reaction of an alcohol and a carboxylic acid. This esterification reaction is also known as a Fischer reaction. The reverse reaction is a hydrolysis. The reactions in both directions are very slow and thus relatively ineffective without the help of a catalyst: a free proton originating either from a strong acid rich in free protons in aqueous solution, or water in which the carboxylic acid is in solution. It is also virtually athermal, that is to say that it neither gives off nor absorbs heat. Thus, a variation in the temperature has no effect on the yield, as is known from the experimental Van't Hoff law. Likewise, a variation in the pressure does not result in a displacement of the equilibrium insofar as the reactants and the products are in most cases liquids, at a constant content of reactants. Only an increase in the temperature accelerates the reaction and makes it possible to reach the esterification equilibrium more quickly.

The conversion of the reaction depends only very slightly on the nature of the carboxylic acid used but depends in particular on the class of the alcohol used. For reactants introduced in equimolar amounts, it is 67% with a primary alcohol, such as methanol, 60% with a secondary alcohol, such as isopropanol, and only 5% if the alcohol is a tertiary alcohol, such as tert-butanol.

In order to increase the yield, it is known to use one of the reactants in excess, generally the cheaper, which will modify the final degree of progression of the reaction and thus the yield. This also makes it possible to shift the equilibrium in the direct direction of esterification, in particular by distilling off the ester as it is formed, if it is more volatile, or by removing the water. For this, it is possible, for example, to use entrainment with an inert substance, a relatively volatile solvent which forms a heteroazeotrope with water being added to the reaction system, or also to incorporate a dehydrating substance in the reaction mixture. However, this presents problems because, on the one hand, even if it is employed in excess, not all the water may be consumed. Furthermore, it is subsequently necessary to separate the ester from this product, which may cause complications and a fall in the yield.

As regards the kinetics, the uncatalyzed esterification reaction is rather slow. The rate also changes according to the class of the alcohols: it decreases on moving from a primary alcohol to a secondary alcohol and then to a tertiary alcohol. The temperature can be increased, so as to greatly improve the kinetics, according to the Arrhenius law, and an acid catalyst can be used which makes it possible to increase the electrophilic nature of the carboxyl group.

The majority of esterification reactions on the industrial scale use a homogeneous acid catalyst, such as sulfuric acid. Mention may be made, to this end, of the publication “Chemie Ingenieur Technik”, 43, 1971, No. 18, 1001-1007, which describes a process for the preparation of esters from alcohols and carboxylic acids in a reactive distillation column using sulfuric acid as homogeneous catalyst. However, this compound is a powerful oxidizing agent which can oxidize the alcohol or even dehydrate it and cause corrosion to the plants.

Furthermore, in addition to the yield and the kinetics, it is necessary to carry out the esterification reaction with, if possible, the best selectivity and a minimum residence time. However, in homogeneous catalysis, high temperatures make it possible for the equilibrium to be established in a reasonable time to promote the most stable product, promoting the kinetics, but at the expense of the selectivity.

Heterogeneous catalysis has made it possible to be unaffected by problems of corrosion of the plants and of selectivity of the esterification reaction, while simplifying the process.

It is known to use heterogeneous catalysts at temperatures generally of between 50° C. and 100° C. as the stability of these catalysts at high temperatures is not established.

Thus, for example, application WO 2007/099071 describes the manufacture of esters, such as the ester ethyl levulinate, by homogeneous or heterogeneous reactive distillation using an extractive substance which makes it possible to entrain, at the column bottom, the ester produced. Use may in particular be made of cyclohexane for this purpose. The catalyst is preferably a sulfonic resin. The process in particular employs the catalyst at a temperature of less than 100° C. and at atmospheric pressure.

U.S. Pat. No. 6,028,215 describes the manufacture of esters, such as the ester butyl acetate, from butanol and acetic acid by heterogeneous reactive distillation. The catalyst is of sulfonic resin type and the ester produced exits at the lower part of the reactive column. The process employs the catalyst at a temperature of between 50 and 160° C. and at a pressure of less than 2 bar.

Application EP 1 220 829 describes the manufacture of the ester ethyl acetate from ethanol and acetic acid by heterogeneous reactive distillation. The ester formed is recovered in the upper part of the reactive column and the catalyst used is of sulfonic resin type. The process described employs the catalyst at atmospheric pressure, and the distillate exits from the column at a temperature in the vicinity of 70° C.

However, the processes described in these documents do not make it possible to obtain an excellent optimum between the conversion, the kinetics and the selectivity of the reaction. There thus existed, in the field of esterification, a need to develop a process which makes it possible to avoid the abovementioned disadvantages and to obtain such a compromise. Furthermore, there existed a need to significantly reduce the production costs for carrying out such esterification processes.

### Invention

It has transpired, entirely surprisingly, that the use of specific heterogeneous acid catalysts in an esterification reaction employing alcohols and carboxylic acids at high temperatures makes it possible to achieve all the abovementioned objectives; in particular to prevent corrosion of the plants while making possible an excellent compromise between the conversion, the kinetics and the selectivity of the reaction.

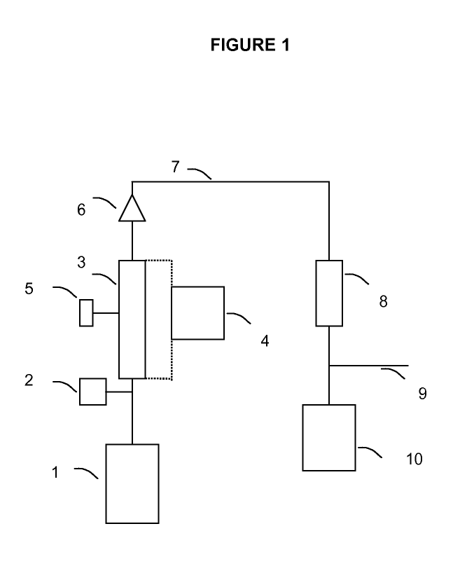

These specific catalysts are identified according to the present invention by their high thermal stability over time during the high temperature esterification reaction. They are characterized by the maintenance of the degree of conversion which they are capable of contributing to this reaction over the complete duration of the test P described below, in particular according to the installation of FIG. 1.

It furthermore transpires that an excellent conversion and selectivity of the reaction are obtained even when using a carboxylic acid/alcohol molar ratio of between 0.5 and 2, that is to say without setting up a high molar imbalance of the reactants.

In addition, it transpires that this increase in temperature used during the esterification reaction can be benefited from economically in the plant to reduce the energy costs, in particular for the subsequent stages of purification of the reactants.

Carrying out the reaction at higher temperatures also makes it possible to reduce the volume necessary for the reaction, which proves to be a great advantage in carrying out the reaction in smaller and thus cheaper installations. Carrying out the reaction at higher temperatures also makes it possible to reduce the amount of catalysts necessary for the reaction, which reduces the costs and the treatment of the effluents.

### DETAILED DESCRIPTION OF THE INVENTION

A first subject matter of the present invention is thus a process of the manufacture of a carboxylic ester by an esterification reaction of a carboxylic acid and an alcohol at a temperature of between 100 and 200° C., in the presence of a heterogeneous acid esterification catalyst;

said catalyst exhibiting a degree of conversion of between 20 and 67%, more preferably between 50 and 67%, throughout the duration of the following test P: an equimolar mixture of acetic acid and ethanol is stored at ambient temperature in a tank (1) positioned on a balance. It is continuously transferred via a volumetric pump (2) at a flow rate of 100 g/h, measured by difference in weighing of the tank on the balance, to a fixed bed reactor (3) comprising approximately 1 g of dry test catalyst. The internal diameter of the bed is 4 mm and its length is 220 mm. The catalyst is held between two screens. The temperature of the reaction is controlled by a thermostatically controlled bath (4) comprising a heat-exchange fluid which circulates in the jacket situated around the reactor. The temperature of the reaction is 140° C. and is monitored by a temperature probe (5). A backpressure valve (6) located on the line (7) at the outlet of the reactor is adjusted to a pressure chosen in order to keep the reaction medium in the liquid phase at the temperature of 140° C. The reaction mixture is reduced in pressure to atmospheric pressure at the outlet of the valve. It is condensed and cooled to 25° C. by an exchanger (8) fed with refrigerated water. A portion of the cooled mixture is sampled via a withdrawal device (9) for chromatographic analysis and the other portion is recovered in the tank (10). The high-temperature activity of the catalyst is monitored by the analysis of the stability of the conversion of the reactants for a duration of operation in continuous mode of 300 h. Such an installation can correspond to that of FIG. 1.

It is then optionally possible to carry out one or more stages of separation of the products resulting from the esterification reaction, so as to isolate the ester formed.

It is optionally possible to benefit economically from the high temperature used during the esterification reaction to reduce or benefit economically from the energy consumption of the whole of the industrial installation, comprising in particular at least one means for carrying out the esterification reaction and at least one means for carrying out the subsequent stage or stages of separation of the reactants. Reduction of or benefiting economically from the energy of the industrial installation is understood to mean in particular the use of the high temperature streams of the esterification reaction stage to heat the low temperature streams used for the subsequent purification stages.

In practice, the high temperature stream exiting, directly or indirectly, from the reactor for the esterification reaction is generally treated in a heat exchanger, such as, for example, a condenser, a flash drum or a reboiler, in order to exchange its energy with means which make it possible to carry out the subsequent stage or stages of separation of the reactants.

The process of the invention can be carried out in various ways and in various types of possible reactors. There is no limitation on the installations which can carry out the reaction of the invention. Detailed information on the possible reactors and installations is given below.

Generally, the process of the invention comprises at least the following stages:

a) feeding at least one alcohol and one carboxylic acid to a reactor;

b) reacting the alcohol and the acid in the presence of a heterogeneous catalyst;

c) carrying out one or more stages of separation of the products resulting from the reaction of stage b), so as to isolate the ester formed; and

d) optionally benefiting economically from the temperature used during stage b) for the separation stage or stages of step c), so as to reduce the energy consumption of this setup.

Several conventional separation processes can be used to separate the reactants, in particular the carboxylic ester, from the other constituents. Mention may be made, for example, as separation process, of distillation, condensation, chromatography, membrane separation and extraction. It is possible to use just one of the separating means or several, continuously or in parallel. It is preferable in particular to operate one or more distillations or else a condensation, followed by one or more distillations.

The temperature of the reaction is preferably between 110 and 180° C. and more preferably between 130 and 170° C.

The pressure of the reaction of the process is preferably between 1 and 20 bar, in particular between 2.5 and 13 bar and more preferably still between 3 and 5 bar.

The reaction of the invention is very preferably carried out at a temperature of between 130 and 170° C. and a pressure of between 3 and 13 bar.

The preferred alcohol is in particular an alcohol comprising from 2 to 6 carbon atoms, such as, in particular, methanol, ethanol, propanol and butanol. Use may in particular be made, during this reaction, of primary or secondary alcohols. Preference is given in particular to alcohols having a low boiling point, such as methanol and ethanol. The carboxylic acid is in particular an acid comprising from 2 to 6 carbon atoms, such as, in particular, acetic acid. The preferred ester is ethyl acetate.

In the process of the invention, the carboxylic acid/alcohol molar ratio can be between 1 and 10.

The heterogeneous acid esterification catalysts of the invention are preferably sulfonic resins. The acid catalysts of the invention can be of the ion-exchange resin type. Use may preferably be made of sulfonic resins having an acidity of between 1 and 10 eq/kg (H⁺). This acidity can be measured, for example, by acid/base titration using sodium hydroxide solution. The particularly preferred catalysts are Amberlyst 70 from Rohm & Haas and Lewatit K2431 from Lanxess. As regards the Lewatit K2431 catalyst, reference may be made to the Lanxess Product Information Sheet of 26/08/08. As regards the Amberlyst 70 catalyst, reference may be made to the Rohm & Haas sheet “Amberlyst™ 70 Strongly Acidic Catalyst”.

For an acid/alcohol equimolar mixture, the concentration of catalyst is generally between 1 and 10% by weight in a slurry stirred reactor, with, for a fixed bed, a contact time between 10 seconds and 1 hour and preferably between 10 seconds and 5 minutes at 140° C.

The esterification reaction between the carboxylic acid and the alcohol can, for example and without any limitation, be carried out in conventional reactors or reactive distillation columns.

The reaction can in particular operate batchwise, continuously or semicontinuously.

The alcohol and the acid can be fed into the reaction region in various ways, for example at the same time by different feed means, or else as a premix, or also in delayed fashion. Use may also be made of pure or virtually pure acid or alcohol or also of mixtures of acid and alcohol comprising other organic or inorganic compounds, such as water. The acid and the alcohol can in particular originate from a prior production or separation means, such as a distillation column.

According to a first alternative form of the invention, the process can be carried out in a conventional reactor in which the heterogeneous catalysts are in suspension (slurry), immobilized on a fixed bed, or on a fluidized bed.

The catalysts can be immobilized at the surface of inert supports, such as metal screens or components made of silica, which form a fixed structure, referred to as fixed bed, or a moving structure, referred to as fluidized bed, placed in the reactor.

Recourse may be had in particular to the reactive adsorption technique in the reactor in order to increase the conversion. An adsorbing agent makes it possible, for example, to remove the water formed during the reaction.

When the process is carried out in a conventional reactor, the carboxylic acid/alcohol molar ratio is preferably between 5 and 10.

It will be preferable to couple, to the conventional esterification reactor, a “primary” distillation column, in order to separate the carboxylic ester from the other constituents of the reaction. In addition, use may also be made of one or more other distillation columns. It is possible, for example, to convey the high-temperature stream from the primary distillation column to a heat exchanger, so as to provide heat to the bottom of the other distillation column or columns.

According to a second alternative form of the invention, use may in particular be made, as installation, of a reactive distillation column which makes it possible to carry out, simultaneously, at least one chemical reaction in the presence of catalyst and the separation by distillation of the reaction mixture obtained. The process and the installation of the invention can be applied to various equilibrated reactions, in the liquid phase, for which the reaction product can be isolated by distillation under the temperature and pressure conditions under which the reaction is carried out.

The reactive distillation column comprises in particular at least one reaction region and at least one nonreaction region.

The packing body optionally used in the process of the present invention is chosen as a function of the efficiency necessary. The packing body can be chosen from the packing bodies well known to a person skilled in the art, such as, for example, solids in the form of rings, polylobal extrudates or saddles. Mention may be made, as nonlimiting examples of packing bodies, of Raschig rings, Pall rings, Intos rings, Berl saddles, Novalox saddles and Intalox saddles. However, the packing body can also be chosen from structured packings, for example of Flexipac (registered trade name) type, sold by Koch, or Sulzer Chemtech or Sulzer (registered trade names) type, sold by Sulzer.

The reaction region of the reactive distillation column generally comprises at least one esterification catalytic bed. The catalyst can be enclosed in at least one permeable casing, which casing is composed, for example, of a cloth made of fabric made of synthetic material, for example polypropylene, or made of metal fabric. The catalyst can also be positioned in bulk, that is to say freely, inside each catalytic bed of the catalytic region. In this case, in order to keep the catalyst in place and to prevent it from being carried away by the stream of liquid which passes through it, provision is generally made for any catalytic bed included in the catalytic region to rest on any contrivance which allows the liquid to pass but which is impermeable to the catalytic particles, such as, for example, a screen of Johnson type or nozzles of Johnson type evenly distributed over a surface impermeable to gas, liquid or solid streams. Advantageously, said contrivance is slightly raised with respect to the inlet of the substantially radial circulation means, over the bottom of the catalytic region, so as to create a distribution region situated partially below the catalytic bed of the catalytic region.

When the catalyst is positioned in bulk, provision may be made to use it in the form of a fixed bed, expanded bed or fluidized bed. Whether the catalyst is positioned in bulk or enclosed in at least one casing, the void fraction which can be conferred on it is generally between 30 and 70%.

As regards the second alternative form using a reactive distillation column, the process generally comprises at least the following stages:

a) feeding at least one alcohol and one carboxylic acid to a distillation column comprising at least one reaction region and at least one nonreaction region;

b) reacting the alcohol and the acid in the reaction region or regions in the presence of a heterogeneous catalyst and separating, by distillation, the compounds formed;

c) carrying out one or more separations of the products resulting from the reaction of stage b), so as to isolate the ester formed; and

d) optionally benefiting economically from the temperature used for the reactive distillation of stage b) in the separation or separations of stage c), so as to reduce the energy consumption of this setup.

The alcohol is preferably fed to the lower part of each reaction region or below each of these regions. The acid is very preferably fed to the upper part of each reaction region or above each of these regions.

It is very particularly preferable for the reaction in stage b) to be carried out so that the carboxylic acid/alcohol molar ratio is between 1 and 2.

When an alcohol of low boiling point, such as ethanol, is used, there is recovered in particular at the top of the reactive distillation column a ternary azeotropic mixture comprising ester, alcohol and water and, at the bottom of the column, the acid and the water. The concentration of ethanol in this azeotropic mixture is generally between 0.5 and 1.5% by weight, preferably in the vicinity of 0.9% by weight. Preference is given in particular to the reaction of acetic acid with ethanol, resulting in the recovery of the carboxylic ester, the water and the unreacted alcohol at the column top and the carboxylic acid at the column bottom.

The proportion of heterogeneous catalyst in the reactive sections is preferably between 10 and 50% by volume.

It will be preferable to couple, to the reactive distillation column, at least one distillation column for separating the carboxylic ester from the other constituents of the reaction mixture. It is possible, for example, to convey the high temperature stream from the esterification reaction stage of the reactive distillation column to a heat exchanger, so as to provide heat to the bottom of this distillation column.

The two alternative forms of the invention mentioned above; that is to say an esterification reaction in a conventional reactor, in particular a fixed bed reactor, and an esterification reaction in a reactive distillation column, can advantageously be combined; for example either consecutively or in parallel. The parallel form is preferable in order to recycle one of the streams exiting from the reactive distillation column to the conventional reactor. The high temperature stream from the reactive distillation column can optionally be used to reduce the energy consumption of the stages of subsequent separations of the reactants. Use may be made in particular of the high temperature stream from the reactive distillation column and from the primary distillation column following the conventional reactor to reduce the energy consumption of the stages of subsequent separations of the reactants.

A specific language is used in the description so as to facilitate understanding of the principle of the invention. Nevertheless, it should be understood that no limitation on the scope of the invention is envisaged by the use of this specific language. Modifications and improvements may in particular be envisaged by a person conversant with the technical field concerned on the basis of his own general knowledge.

The term and/or includes the meanings and, or, and also all the other possible combinations of the elements connected to this term.

Other details and advantages of the invention will become more clearly apparent in the light of the examples given below solely by way of indication.

Experimental Part

### EXAMPLE 1

### Simple Reactor

Ethyl acetate (RN-CAS 141-78-6) is conventionally synthesized by esterification of acetic acid (RN-CAS 64-19-7) with ethanol (RN-CAS 64-17-5). This athermal and equilibrated reaction is accelerated by heterogeneous acid catalysis with acidic potential catalysts, such as sulfonic resins.

Two resins are used in this experimental part:
the resin A70, sold by Rohm & Haas (degree of conversion of 67% according to the test P)
the resin K2431, sold by Lanxess (degree of conversion of 67% according to the test P).

After washing 4 times with demineralized water, with 2 volumes of water per 1 volume of wet resin, and then washing 4 times with absolute ethanol, with 2 volumes of alcohol per 1 volume of wet resin, each of these two resins can be used in a chemical reaction.

The change in kinetics of the reaction under acid/alcohol equimolar conditions, with 5% by weight of resin A70, in a stirred reactor under a pressure of 12 bar, at respectively 70 and 160° C., is shown in table 1 below. For each resin, the energy of activation of the direct and reverse reactions is estimated at 50 kJ.mol⁻¹.

TABLE 1

| | Weight of ethyl | Weight of ethyl |
|---|---|---|
| Reaction time | acetate at 70° C. | acetate at 160° C. |
| (min) | (g) | (g) |
| 0 | 0 | 0 |
| 10 | 20 | 60 |
| 20 | 32 | 60 |
| 30 | 40 | 60 |
| 40 | 45 | 60 |
| 50 | 50 | 60 |
| 60 | 51 | 60 |

The change in kinetics of the reaction under acid/alcohol equimolar conditions, with 5% by weight of resin K2431, in a stirred reactor under a pressure of 12 bar, at respectively 70 and 160° C., is shown in table 2 below. For each resin, the energy of activation of the direct and reverse reactions is estimated at 50 kJ.mol⁻¹.

TABLE 2

| | Weight of ethyl | Weight of ethyl |
|---|---|---|
| Reaction time | acetate at 70° C. | acetate at 160° C. |
| (min) | (g) | (g) |
| 0 | 0 | 0 |
| 10 | 25 | 60 |
| 20 | 37 | 60 |
| 30 | 45 | 60 |
| 40 | 50 | 60 |
| 50 | 52 | 60 |
| 60 | 54 | 60 |

Despite the high temperature level, no predominant undesired chemistry is observed on the chromatograms obtained by conventional GC analysis with a dilution of the crude reaction product by a factor of 200 by volume.

1. A process of manufacturing a carboxylic ester, the process comprising performing by an esterification reaction of a carboxylic acid and an alcohol at a temperature of between 100° C. and 200° C., with a heterogeneous acid esterification catalyst being present;
said catalyst exhibiting a degree of conversion of between 20% and 67%, throughout the duration of the following test P: storing an equimolar mixture of acetic acid and ethanol at ambient temperature in a tank (1) positioned on a balance, continuously transferring the tank via a volumetric pump (2) at a flow rate of 100 g/h, measured by difference in weighing of the tank on the balance, to a fixed bed reactor (3) comprising approximately 1 g of dry test catalyst, wherein the internal diameter of the bed is 4 mm and its length is 220 mm, holding the catalyst between two screens controlling the temperature of the reaction with a thermostatically controlled bath (4) comprising a heat-exchange fluid which circulates in a jacket situated around a reactor, wherein the temperature of the reaction is 140° C. and is monitored by a temperature probe (5), wherein a backpressure valve (6) is located on line (7) at an outlet of the reactor and is adjusted to a pressure chosen in order to keep the reaction medium in a liquid phase at the temperature of 140° C., reducing the reaction mixture in pressure to atmospheric pressure at an outlet of the valve, condensing and cooling the mixture to 25° C. by an exchanger (8) fed with refrigerated water, sampling a portion of the cooled mixture via a withdrawal device (9) for chromatographic analysis and recovering another portion in a tank (10), monitoring high-temperature activity of the catalyst by analyzing stability of the conversion of the reactants for a duration of operation in continuous mode of 300 h.

2. The process as claimed in claim 1, wherein the process further comprises at least the following stages:
a) feeding at least one alcohol and one carboxylic acid to a reactor;
b) reacting the alcohol and the acid in the presence of a heterogeneous catalyst;
c) carrying out one or more stages of separation of the products resulting from the reaction of stage b), so as to isolate the ester formed; and
d) optionally benefiting economically from the temperature used during stage b) for the separation stage or stages of step c), which reduces energy consumption during the process.

3. The process as claimed in claim 1, wherein the temperature of the reaction is between 130° C. and 170° C.

4. The process as claimed in claim 1, wherein the pressure of the reaction is between 1 bar and 20 bar.

5. The process as claimed in claim 1, wherein the esterification reaction is carried out at a temperature of between 130° C. and 170° C. and a pressure of between 3 bar and 13 bar.

6. The process as claimed in claim 1, wherein the alcohol is selected from the group consisting of methanol, ethanol, propanol and butanol.

7. The process as claimed in claim 1, wherein the carboxylic acid is acetic acid.

8. The process as claimed in claim 1, wherein the ester formed is ethyl acetate.

9. The process as claimed in claim 1, wherein the carboxylic acid/alcohol molar ratio used in the esterification reaction is between 1 and 10.

10. The process as claimed in claim 1, wherein the heterogeneous catalyst is a sulfonic resin.

11. The process as claimed in claim 1, wherein the heterogeneous catalyst is a sulfonic resin having an acidity of between 1 eq/kg H⁺ and 10 eq/kg H⁺.

12. The process as claimed in claim 1, wherein the heterogeneous catalyst is Amberlyst 70 or Lewatit K2431.

13. The process as claimed in claim 1, wherein the esterification reaction is carried out in a conventional reactor exhibiting catalysts in suspension, immobilized on a fixed bed, or on a fluidized bed.

14. The process as claimed in claim 1, wherein the esterification reaction is carried out in a reactive distillation column.

15. The process as claimed in claim 14, further comprising at least the following stages:
a) feeding at least one alcohol and one carboxylic acid to a distillation column comprising at least one reaction region and at least one nonreaction region;
b) reacting the alcohol and the acid in a reaction region or regions in with a heterogeneous catalyst and separating, by distillation, compounds formed;
c) carrying out one or more separations of the compounds resulting from the reaction of stage b), so as to isolate the ester formed; and
d) optionally benefiting economically from the temperature used for the reactive distillation of stage b) in the separation or separations of stage c), which reduces energy consumption of the process.

16. The process as claimed in claim 14, wherein the carboxylic acid/alcohol molar ratio used in the reaction is between 1 and 2.

17. The process as claimed in claim 14, wherein the process comprises reacting acetic acid with ethanol, resulting in recovery of a carboxylic ester, water and unreacted alcohol at a top of a column and a carboxylic acid at a bottom of the column.

18. The process as claimed in claim 1, wherein an esterification reaction is carried out in a conventional reactor and an esterification reaction is carried out in a reactive distillation column, either consecutively or in parallel.
